(19) United States
(12) Patent Application Publication
(10) Pub. No.: US 2004/0009467 A1
(43) Pub. Date: Jan. 15, 2004

(54) ACE-pigments and humic acid as energy sources

(76) Inventor: William John Martin, South Pasadena, CA (US)

Correspondence Address: W. John Martin, 1634 SPRUCE STREET, South PASADENA, CA, 91030, US

(21) Appl. No.: 10/192,936

(22) Filed: Jul. 10, 2002

(51) Int. Cl. C12Q 1/70; A61K 35/78

(52) U.S. Cl. 435/5; 60/721

(57) ABSTRACT

Mineral-containing, auto-fluorescent, pigmented materials are disclosed that comprise conglomerates or aggregates of very fine chemical structures that are held together by a newly defined force termed Selective Mineral Affinity Force (SMAF). Particles comprising these materials can form on the skin of some stealth virus infected patients. They can also be seen attached to the hair strands of such patients. Similar particles form in stealth virus cultures and are thought to provide an alternative (non-mitochondria) cellular energy source for the virally infected cells, including the conversion of electromagnetic energy into chemical energy. These particles have been called alternative cellular energy (ACE)-pigments. The patient skin and hair derived aggregated ACE-pigments can be disrupted into highly energetic smaller components using various chemicals, including aldehydes and alcohols. Humic acid which is commonly used to enhance plant growth and occasionally also used as a health-promoting food supplement for human use, was disclosed as also comprising a conglomerate of tightly attached components that could also by readily disrupted by these same chemicals. The present invention relates to the detection, characterization and the use of ACE-pigments and humic acid as a source of SMAF and also as a potential means of converting electromagnetic energy into chemical energy.

COPENDING with application Ser. Nos. 10/044,683, 10/050,232, 10/058,480, 10/047,313, 10/______ (see below) and 10/______ (see below).

### CROSS REFERENCE TO RELATED APPLICATION

United States Patents

Awarded:

U.S. Pat. No. 5,985,546 Stealth virus detection in the chronic fatigue syndrome. William John Martin

U.S. Pat. No. 5,891,468 Stealth virus detection in the chronic fatigue syndrome. William John Martin

U.S. Pat. No. 5,753,488 Isolated stealth viruses and related vaccines. William John Martin

U.S. Pat. No. 5,703,221 Stealth virus nucleic acids and related methods. William John Martin

Pending:

Ser. No. 10/044,683. Therapy of stealth virus associated cancers and other conditions using light. William John Martin.

Ser. No. 10/047,313. Therapy of stealth virus associated cancers and other conditions using medium chain triglycerides. William John Martin.

Ser. No. 10/050,232. Diagnosing and monitoring the therapy of stealth virus infections based on the detection of auto-fluorescent material in hair. William John Martin.

Ser. No. 10/058,480. Therapy of stealth virus associated cancers and other conditions using magnetic energy. William John Martin.

10/______ (copending) Energy supportive therapy of stealth virus associated diseases. William John Martin.

10/______ (copending) Sound therapy of stealth virus associated diseases. William John Martin.

PCT (Patent Cooperation Treaty)

WO 92/20797 Stealth virus detection in the chronic fatigue syndrome

WO 98/27744 Stealth virus nucleic acids and related methods

WO 99/34019 Stealth virus nucleic acids and related methods

WO 99/60101 Stealth viruses and related vaccines

### REFERENCES TO PUBLISHED ARTICLES

Stealth Viruses:

1 Martin W J Chronic fatigue syndrome among physicians. A potential result of occupational exposure to stealth viruses. Explore 2001; 10 (5): 7-10.

2 Martin W J. Stealth Viruses. Explore 2001; 10 (4): 17-19.

3 Durie G M, Collins R. Martin W J. Positive stealth virus cultures in multiple myeloma. A possible explanation for neuropsychiatric co-morbidity. Presented at the Am. Soc. Hematology annual meeting October 2000.

4 Martin W J. Chemokine receptor-related genetic sequences in an African green monkey simian cytomegalovirus-derived stealth virus. Exp Mol Pathol. 2000; 69:10-6.

5 Martin W J. and Anderson D. Stealth virus epidemic in the Mohave Valley: Severe vacuolating encephalopathy in a child presenting with a behavioral disorder. Exp Mol Pathol. 1999; 66:19-30.

6 Martin W J. Melanoma growth stimulatory activity (MGSA/GRO-alpha) chemokine genes incorporated into an African green monkey simian cytomegalovirus-derived stealth virus. Exp Mol Pathol. 1999; 66:15-8.

7 Martin W J. Bacteria-related sequences in a simian cytomegalovirus-derived stealth virus culture. Exp Mol Pathol. 1999; 66:8-14.

8 Martin W J. Stealth adaptation of an African green monkey simian cytomegalovirus. Exp Mol Pathol. 1999; 66:3-7.

9 Martin W J. Cellular sequences in stealth viruses. Pathobiology 1998; 66:53-8.

10 Martin W J. Detection of RNA sequences in cultures of a stealth virus isolated from the cerebrospinal fluid of a health care worker with chronic fatigue syndrome. Case report. Pathobiology. 1997; 65:57-60.

11 Martin W J. and Anderson D. Stealth virus epidemic in the Mohave Valley. I. Initial report of virus isolation. Pathobiology. 1997; 65:51-6.

12 Martin W J. Simian cytomegalovirus-related stealth virus isolated from the cerebrospinal fluid of a patient with bipolar psychosis and acute encephalopathy. Pathobiology. 1996; 64:64-6.

13 Martin W J. Stealth viral encephalopathy: report of a fatal case complicated by cerebral vasculitis. Pathobiology. 1996; 64:59-63.

14 Martin W J. Genetic instability and fragmentation of a stealth viral genome. Pathobiology. 1996; 64:9-17.

15 Martin W J. Severe stealth virus encephalopathy following chronic-fatigue-syndrome-like illness: clinical and histopathological features. Pathobiology. 1996; 64:1-8.

16 Martin W J. Stealth virus isolated from an autistic child. J Autism Dev Disord. 1995; 25:223-4.

17 Gollard R P, Mayr A., Rice D A, Martin W J. Herpesvirus-related sequences in salivary gland tumors. J Exp Clin Cancer Res., 1996; 15: 1-4.

18 Martin W J. and Glass R T. Acute encephalopathy induced in cats with a stealth virus isolated from a patient with chronic fatigue syndrome. Pathobiology. 1995; 63:115-8.

19 Martin W J, et al. African green monkey origin of the a typical cytopathic ‘stealth virus’ isolated from a patient with chronic fatigue syndrome. Clin Diag Virol 1995: 4: 93-103.

20 Martin W J. Stealth viruses as neuropathogens. CAP Today. 1994; 8: 67-70.

21 Martin W J. et al. Cytomegalovirus-related sequence in an a typical cytopathic virus repeatedly isolated from a patient with chronic fatigue syndrome. Am J Pathol. 1994; 145: 440-51.

22 Martin W J. Stealth adapted viruses and viteria. Insights into virus cionstruction, replication and potential therapies based on DNA sequence analysis of an African green monkey simian cytomegalovirus-derived stealth virus. Abstract in Proceedings International Conference on Emerging Infectious Diseases. Atlanta Ga. March 2002.

### OTHER REFERENCES

Ait-Ameur A, Bern P, Firoloni M P, Menecier P. Delusional parasitosis or Ekbom's syndrome. Rev Med Interne 2000;21:182-6.

Hertkorn N, Permin A, Perminova I, Kovalevskii D, Yudov M, Petrosyan V, Kettrup A. Comparative analysis of partial structures of a peat humic and fulvic acid using one- and two-dimensional nuclear magnetic resonance spectroscopy. J Environ Qual 2002;31:375-87.

Proia A D, Brinn N T. Identification of calcium oxalate crystals using alizarin red S stain. Arch Pathol Lab Med 1985;109:186-9.

Simpson A J, Kingery W L, Hayes M H, Spraul M, Humpfer E, Dvortsak P, Kerssebaum R, Godejohann M, Hofmann M. Molecular structures and associations of humic substances in the terrestrial environment. Naturwissenschaften. 2002;89:84-8.

### BACKGROUND OF THE INVENTION

I have previously described the tissue culture production of pigmented particulate materials in viral cultures from patients infected with stealth-adapted viruses. The materials develop within clusters of virally infected cells, and can also be released into the extra-cellular tissue culture medium. Although, they are clearly heterogeneous, these materials have certain common features. In particular, the materials are pigmented, auto-fluorescent, occasionally magnetic, and their production can be increased by exposing the cultures to light, x-ray and gamma ray electromagnetic radiation. X-ray spectroscopic analysis of several of the free floating extra-cellular particles showed that they differed in their compositions, but typically contained varying proportions of various minerals, including metals. Purified pigmented material was also able to mediate oxidation-reduction reactions, such as the reduction of the yellow colored indicator tetrazolium salt, MTT, to blue formazan.

Tissues from stealth virus infected subjects show a typical inclusions that probably reflect the in vivo counterparts of the abnormal materials being produced in the viral cultures. Hair obtained from stealth virus infected patients can also contain material that will auto-fluorescence and which can have a high content of various minerals, including metals. An incidental observation made while examining hair samples was the occurrence of occasional brightly auto-fluorescent small particulate deposits tightly adhering to the external surface of the hair strands.

Cells normally derive their chemical energy for mitochondria. One of the notable features on stealth virally infected cells is the marked swelling and morphological disruption of the cells' mitochondria. As this research progressed, I recognized the potential linkage between the damage occurring to the mitochondria and the production of the pigmented auto-fluorescent materials by infected cells. Specifically, I postulated that the aberrant materials accumulating in stealth virus infected cells were a compensatory means of providing an alternative (non-mitochondria) source of cellular energy. Moreover, it appeared that these materials were capable of converting electromagnetic energies into chemical energy. I coined the term alternative cellular energy (ACE)-pigments for these materials.

The finding of small particles attached to hair was interesting since stealth virus infected patients occasionally mention abnormal dark skin flakes coming from their scalp. These are sometimes misidentified as products from lice, leading to treatment recommendations that can prove to be quite toxic for these patients. I have also seen unusual pigmented lesions on the skin of stealth virus infected dogs. While I was able to reassure the owners that they were not tumors, I was uncertain of their true nature. Psychiatrists occasionally encounter patients who believe they are infested with skin parasites, a condition known as Ekbom's syndrome or delusional parasitosis (Ait-Ameur A, et al. 2000). Upon interviewing some of these patients, I understood they were both feeling and seeing rough granular substances on their skin. When a recent patient mentioned he was shedding unusual particles onto his bed sheets, I asked that he collect all of particles released over a three day period and to send them to me. The particular patient had a strongly positive stealth virus culture and his hair had also shown prominent auto-fluorescence.

Certain deposits of partially decomposed pigmented plant material contain a complex substance known as humic acid (Hertkorn N. et al. 2002). These deposits exists especially in previously heavily forested areas with moist, swamp-like conditions. The deposits represent a stage between decaying vegetation (humus/humates/peat) and eventual potential formation of coal and oil. Although quite variable, humic acid containing deposits are regarded as an excellent source of micro-nutrients for enhancing the growth and metabolic functions of plants, especially in nutrient deficient soils. Humic acid contains a diverse array of relatively low molecular weight entities including metals, aliphatic acids, ethers, esters, alcohols, aromatic lignin derived fragments, polysaccharides and polypeptides (Simpson A J et al. 2002). Humic acid has also been sold as a dietary supplement for human use. It can also be applied directly to the skin, a practice common in European health spas.

Humic acid is typically extracted from humus on the basis of its solubility in strong alkali and subsequent precipitation in strong acid. The remaining solubilized material is a somewhat refined version of humic acid, referred to as fulvic acid. Other terms that are used for humic acid include humin and moor. Soluble preparations of humic acids are also being marketed, especially as plant food supplements.

The present patent application provides parallel observations that functionally link the pigmented materials produced in response to stealth virus infection to the humic acid substances obtained from the soil. This unanticipated association opens up many exciting possibilities regarding the nutritional support of stealth virus infected cells and an enhanced understanding of the nature and potential energy uses of ACE-pigments and the much more widely available substances generally grouped under the term humic acid.

Methods for the detection and characterization of stealth viruses are covered in United States patents. The formation of auto-fluorescent and magnetic materials in stealth virus cultures has been described in recent patent applications (Ser. Nos. 10/044,683, 10/050,232 and 10/058,480). Therapeutic methods based on the presence of these materials in stealth virus infected cells are disclosed in copending patent application Ser. Nos. 10/047,313, 10/044,683, 10/050,232, 10/058,480, 10/______ and 10/______. Stealth virus-related references are listed in this application and are numbered 1-22. These awarded and pending patents and the entire list of stealth virus publications are incorporated herein by reference.

### BRIEF SUMMARY OF THE INVENTION

The present invention discloses morphological and functional relationships between ACE-pigments produced by stealth viral infected patients, and humic acid derived from decaying vegetation. Cultures of stealth-adapted viruses will commonly show the production of pigmented cell clusters. Although heterogeneous, the pigmented materials are generally highly auto-fluorescent and some clusters display ferro-magnetic properties when approached with a magnet. I have termed these materials alternative cellular energy (ACE)-pigments since they can be activated by various forms of electromagnetic radiation, and presumably can use this energy to provide a non-mitochondria source of chemical energy to infected cells. This application discloses that similar pigmented particulate materials can collect on the skin of stealth virus infected patients and can also be seen attached to strands of hair. Of importance is the finding that individual pigmented particles differ in the relative content of various metal and non-metal elements detectable using x-ray spectroscopy. The present invention also discloses that the particles obtained from patients and humic acid particles comprise aggregates (or conglomerates) of numerous smaller components. These smaller components can be released in a highly energized state by exposure of the larger particles to certain aldehydes and alcohols. The smaller components tend to re-aggregate in a selective manner, presumably based in part on their composition. Selective re-aggregation can also be observed with dispersed humic acid material. The process of re-aggregation is mediated by a force that is distinct from either electrical or magnetic energy and that has been provisionally termed Selective Mineral Affinity Force (SMAF). The present patent application discloses that ACE-pigments and humic acid are potential sources of SMAF and that this energy is likely to find use in various applications that currently depend on electrical or magnetic energies. ACE-pigments and humic acid can also be utilized as a source of chemical energy, and as sensors for electromagnetic radiation.

### BRIEF DESCRIPTION OF THE DRAWINGS

The accompanying drawings, which are incorporated in and constitute a part of the specification, illustrate the various findings described in this application. They serve to explain the foundations and the principles of the invention.

FIG. 1. Photographs of many of the pigmented particles received from the patient. A 60 millimeter (mm) ruler is shown for size comparison.

FIG. 2. Photomicrograph of one of the particle from the patient as seen using a 4× objective and a 10× eyepiece. The particle was in a drop of medium that had been placed on a microscope slide.

FIG. 3. Photomicrograph of the same particle as shown in FIG. 2. The particle had been rotated 90° by approaching the microscope slide with a small hand held magnet.

FIG. 4. Photomicrograph of the same particle as shown in FIG. 3. The particle had been rotated an additional 90° by repositioning the magnet.

FIG. 5. Combined green and red auto-fluorescence of a particle obtained from the patient when examined using confocal microscopy. Although much of the photograph shows confluent areas of whiteness, when actually observed most of these areas were very closely spaced pin-points of very bright red and green auto-fluorescence.

FIG. 6. Auto-fluorescence of another particle obtained from the patient when examined using confocal microscopy. It showed many of the same types of very small areas of auto-fluorescence as was seen in FIG. 5. In addition several rather larger rounded areas of uniform auto-fluorescence could be seen.

FIG. 7. Auto-fluorescence of another particle obtained from the patient. This photomicrograph was included to show the irregularity of the auto-fluorescent patches seen with some of the particles. Some of the patches of auto-fluorescence showed quite different levels of intensity depending upon whether a blue or a green excitation light was being used.

FIG. 8. Auto-fluorescence of another particle that showed a relatively lower level of auto-fluorescence than did most of the other particles examined. examined by confocal fluorescent microscopy.

FIG. 9. Low magnification scanning electron microscopic appearance of number 1 of 6 particles that were examined by x-ray spectroscopy. A size marker is included in the photograph.

FIG. 10. A higher magnification of particle number 1 showing more details of the surface topography. The black area is a hole that was present in the particle.

FIG. 11. Low magnification scanning electron microscopic appearance of number 2 of 6 particles that were examined by x-ray spectroscopy.

FIG. 12. A higher magnification of particle number 2 showing more details of the surface topography. The very bright areas were emitting more x-rays than was the less brightly staining regions.

FIG. 13. Low magnification scanning electron microscopic appearance of number 3 of 6 particles that were examined by x-ray spectroscopy.

FIG. 14. A higher magnification of particle number 3 showing more details of the surface topography.

FIG. 15. Low magnification scanning electron microscopic appearance of number 4 of 6 particles that were examined by x-ray spectroscopy.

FIG. 16. A higher magnification of particle number 4 showing more details of the surface topography.

FIG. 17. Low magnification scanning electron microscopic appearance of number 5 of 6 particles that were examined by x-ray spectroscopy.

FIG. 18. A higher magnification of particle number 5 showing more details of the surface topography.

FIG. 19. Low magnification scanning electron microscopic appearance of number 6 of 6 particles that were examined by x-ray spectroscopy. The cracks seen in this particle were caused by the vacuum used in the scanning electron microscope

FIG. 20. A higher magnification of particle number 6 showing more details of the surface topography.

FIG. 21. Photomicrograph of the particle shown in FIGS. 2-4. The particle had attached itself to another particle that also showed magnetic activity. A gas bubble can be seen that had come from the larger particle.

FIG. 22. A positive MTT reduction assay occurring around a particle. The dark material is formzan that develops from the yellow tetrazolium salt, MTT.

FIG. 23. Patchy auto-fluorescence seen with a humic acid particle. The photograph combines the patterns seen with blue and green light excitation. When individually examined, most of the areas could be shown to be responsive to both blue and green excitation. There were some areas, however, that showed a more selective response.

FIG. 24. Auto-fluorescence of two other humic acid particles showing patches of auto-fluorescence of varying intensities.

FIG. 25. Low magnification scanning electron microscopic appearance of a humic acid particle that was examined by x-ray spectroscopy. A size marker is included in the photograph.

FIG. 26. A higher magnification of the humic acid particle showing the roughly surfaced, varying sized, conglomeration (breccia) of multiple components.

FIG. 27. Photograph of a flask showing the aggregation pattern that developed from what had been a very evenly distributed extremely fine flocculent material that was precipitated from a soluble preparation of humic acids by the addition of 20% acetic acid.

FIG. 28. The centralized coalescence pattern that developed from many small humic acid particles spread evenly onto the surface of water in a Petri dish. The various particles moved about each other while generally moving towards the center. Progressively the different particles would come into closer contact and show selective adherence to form the type of complex pattern shown. Some of the particles from the center had sunk to the bottom of the Petri dish.

FIG. 29. Coalescence of humic acid particles that were initially evenly distributed on the bottom of a Petri dish, left out in the sun. The outside ring of particles had formed well before the fluid finally evaporated. Using a magnifying glass, it was possible to directly observe significant movements and displacements of many of the particles as they began to form into linear steaks and rounded clusters.

FIG. 30. A scanning electron micrograph of a particle seen attached to one of the hair strands obtained from a stealth virus positive patient. A size marker is shown. The cross indicates the area from which the x-ray spectroscopy reading was taken.

FIG. 31. X-ray spectroscopy analysis of the particle shown in FIG. 30. It shows a predominance of sulfur and calcium.

FIG. 32. A scanning electron micrograph of another particle seen tightly attached to one of the hair strands obtained from a stealth virus positive patient. A size marker is shown. The cross indicates the area from which the x-ray spectroscopy reading was taken.

FIG. 33. X-ray spectroscopy of particle shown in FIG. 32. It shows a predominance of sulfur, chromium and iron with lesser amounts of silicon, nickel and copper.

FIG. 34. A scanning electron micrograph of another particle seen tightly attached to one of the hair strands obtained from a stealth virus positive patient. A size marker is shown. The cross indicates the area from which the x-ray spectroscopy reading was taken.

FIG. 35. X-ray spectroscopy of particle shown in FIG. 34. It shows the same elements as in the particle analyzed in FIG. 33 but in different ratios.

FIG. 36. A scanning electron micrograph showing two adjacent particles tightly attached to a hair strand. The cross indicates that the next analysis was to be performed on the lower of the two particles.

FIG. 37. X-ray spectroscopy of lower particle indicated in FIG. 36. It shows predominantly sulfur with a very small amount of calcium.

FIG. 38. A scanning electron micrograph showing the same two adjacent particles as shown in FIG. 37. The cross indicates that the next analysis was to be performed on the upper of the two particles.

FIG. 39. X-ray spectroscopy of upper particle indicated in FIG. 38. It shows a complex pattern with small amounts of aluminum, silica, sulfur and chromium, but with considerable amount of iron and a striking amount of nickel. The unshaded peak to the left of this Figure also represents nickel (K shell).

### DETAILED DESCRIPTION OF THE INVENTION

The patient material was received in a plastic Ziploc™ (S C Johnson and Sons, Inc.) covered bowel 4″ wide at its top, 2″ wide at its base and 2″ high. It contained 42 irregularly shaped, small stone-like particles, varying in size from approximately 0.5 to 5 mm with an overall weight of 52 mg. The patient was unaware of any skin lesions that could account for the passage through his skin of any of the material as intact particles. He had noted, however, that some of the smaller particles tended to cling together on the bed sheets.

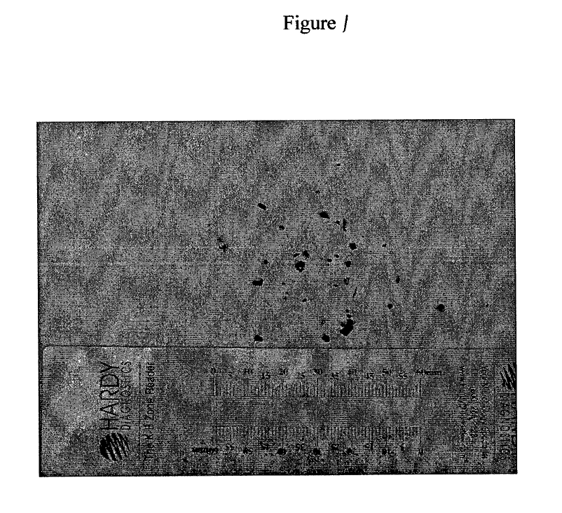

The particles generally had a dark brownish color, but several were of a distinctly black color, others of a lighter brown color, and one with a somewhat reddish color. A photograph of some of the particles placed near a metric ruler is shown in FIG. 1.

I first confirmed that the grouping of particles had an electrostatic charge. I did so before even opening the Ziploc™ container. By briefly rubbing a plastic ruler (Newell Company, Freeport Ill.) against the clothing between my arm and chest, I could create an electrostatic negative charge on its surface. I positioned the particles to one side of the Ziploc™ container. I then compared the attraction of the ruler for the container in the region when the particles were present versus the other side. There was very apparent final few millimeter active attraction of the ruler towards, and discernable sticking of the ruler to, the side of the container adjacent to the particles. This observation contrasted with a clear lack of attraction of the rubbed ruler when applied to the opposite side of the container. This simple experiment confirmed that the particles could established an electrostatic field. I could further confirmed this by taking several particles and placing them on a table. Some of them sprang 1-2 inches off the table to adhere to an approaching, recently-rubbed, ruler. In one striking example, the particle stayed momentarily attached to the ruler, partially rotated and then shot back to the table, only to spring back up the ruler, and then down, up and down again. It then essentially lost its electrostatic charge and could no longer even bind to a freshly rubbed ruler. The most plausible explanation is that the particle had an initial positive charge. Upon contact with the ruler, it was able to acquire a negative charge from the ruler. It was thereupon repelled by the ruler. It then gave up the acquired negative charge to the table, and the process repeated itself until the particle had essentially fully discharged all of its inherent positive electrostatic potential.

I could similarly demonstrate magnetic activity of some of the particles. A hand held magnet approaching the container caused clearly discernable movement of a minority (estimated ⅕) of the particles. When laid out on a sheet of paper it was clear that even these reactive particles differed in the strength of their magnetic activity. Some were very responsive to an approaching magnet, while other required virtual touching with the magnet to become adherent. With several particles, the induced movement was alternatively attraction and repulsion depending upon the pole of the approaching magnet. This indicated true ferro-magnetic activity, and not simply paramagnetic activity.

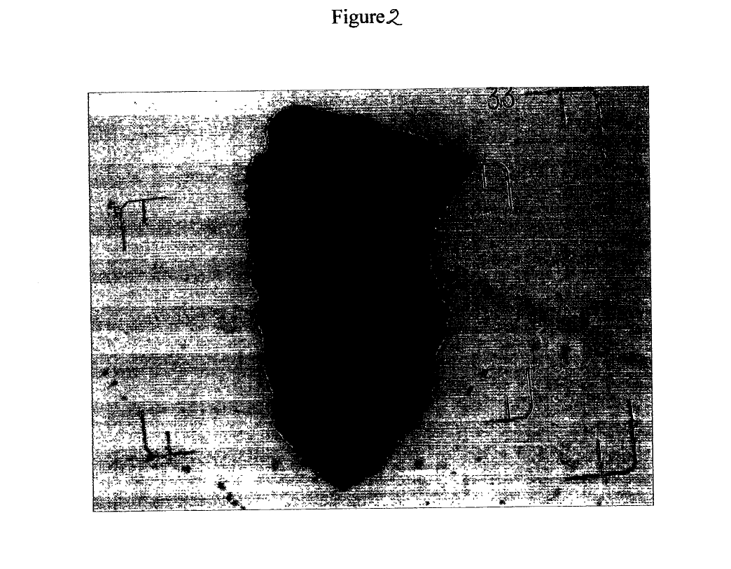

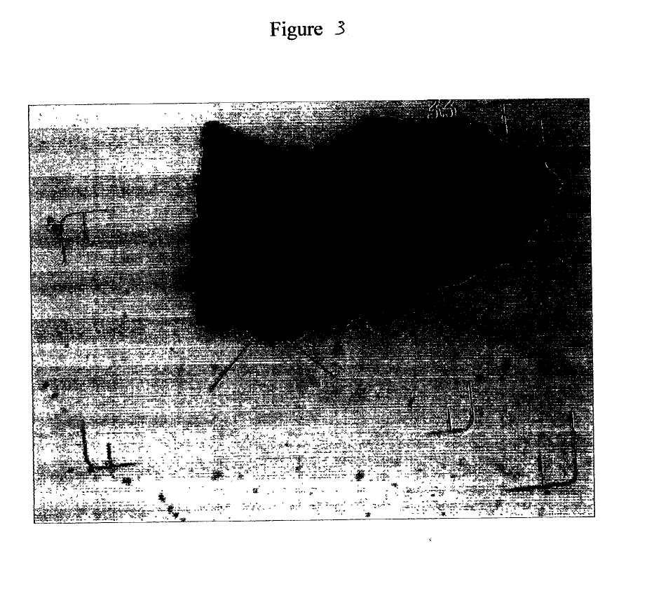

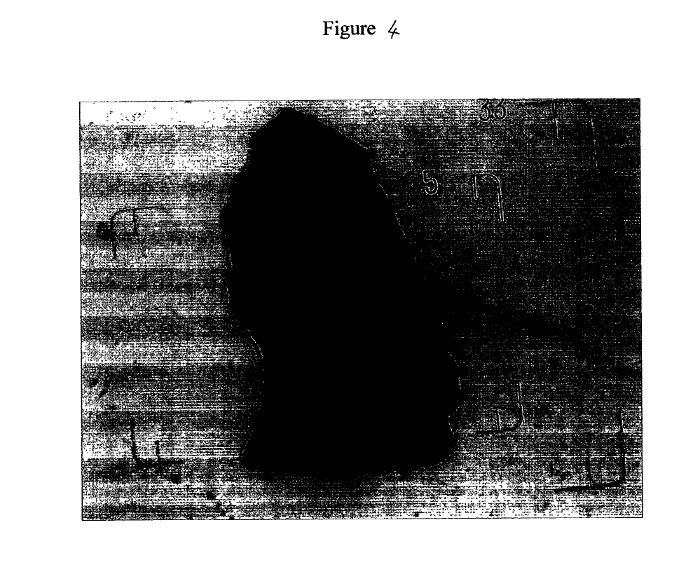

I placed several particles into a drop of X Vivo-15 tissue culture medium on a microscope slide and looked at the slide with a microscope. I positioned one of the particles into the center of the microscopic field of view (FIG. 2). By approaching the slide with a magnet, I could easily rotate the particle at will. Photomicrographs of the same particle shown in FIG. 2, but rotated at 90° and 180° by a hand held magnet are provided in FIGS. 3 and 4, respectively.

An interesting observation was the loss of both electrostatic and magnetic activity of many of the particles following several minutes of using the magnet and/or rubbed ruler. A few particles, however, retained strong magnetic activity. By allowing for a delay in exposure to the magnet, some of the original magnetic activity returned. I subsequently also learned that I could reestablish electrostatic activity in some of the particles by exposing them to bright sunlight for several minutes.

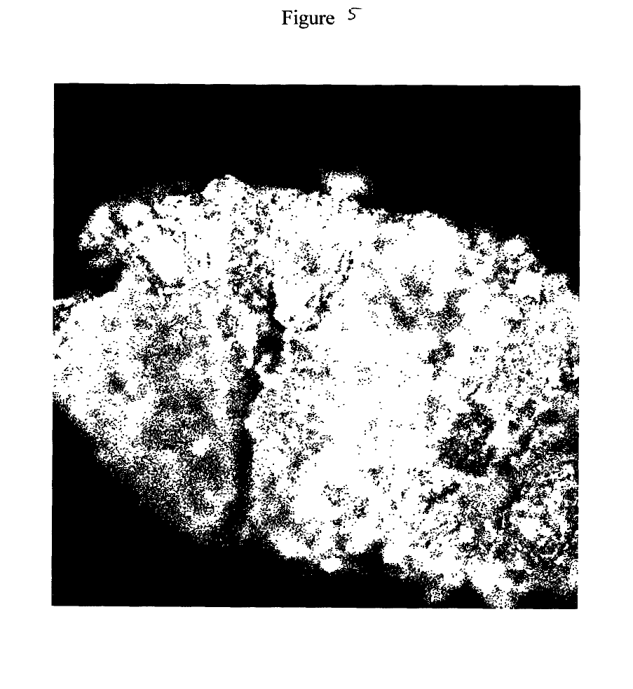

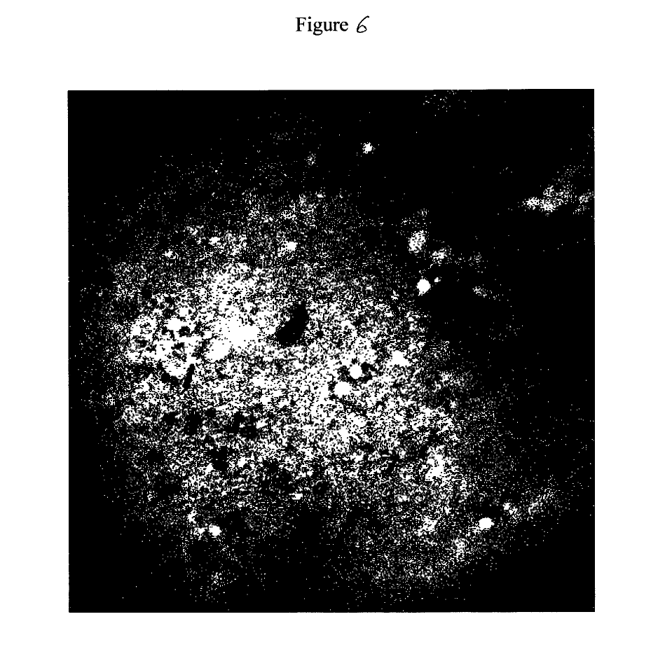

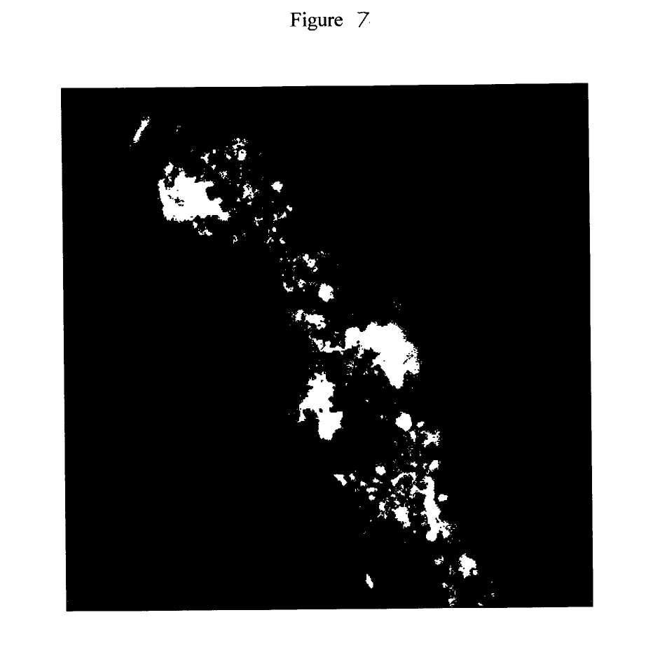

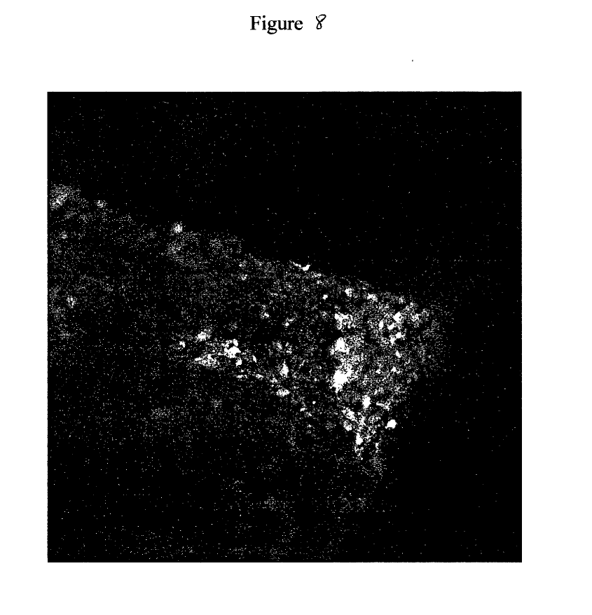

Representative particles from the patient were next examined for auto-fluorescence. Under blue light illumination, I could sense that the particles were comprised of a very large number of irregularly contoured smaller components. There was no sense of order of the components, as would be expected in a uniformly structured crystal. Rather, the varying components differed considerably in size, shape and in both the color and intensity of the blue light induced auto-fluorescence. I repeated this study using a dual laser confocal fluorescent microscope, available at the City of Hope Medical Center, Duarte Calif. With both blue (488 nm wavelength) and green (543 nm wavelength) excitation, numerous, varying sized patches of very bright, sustainable auto-fluorescence were readily observed. The red fluorescence, evoked by the green laser light, was generally brighter, and somewhat more widely distributed than the green auto-fluorescence evoked by the blue laser light. Within a single particle, I could occasionally see patches that would show exclusively either a red or a green auto-fluorescence. Overall, the confocal microscopist (Mr. Bob Barber) and I agreed on my earlier impression that the particles were composed of a conglomerate of numerous, variously sized, minor components, with varying auto-fluorescence properties. Examples of auto-fluorescence are provided in FIGS. 5-8.

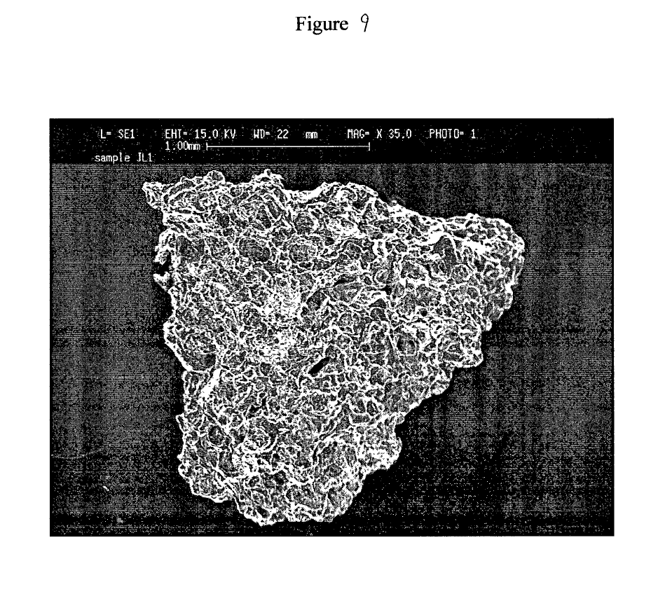

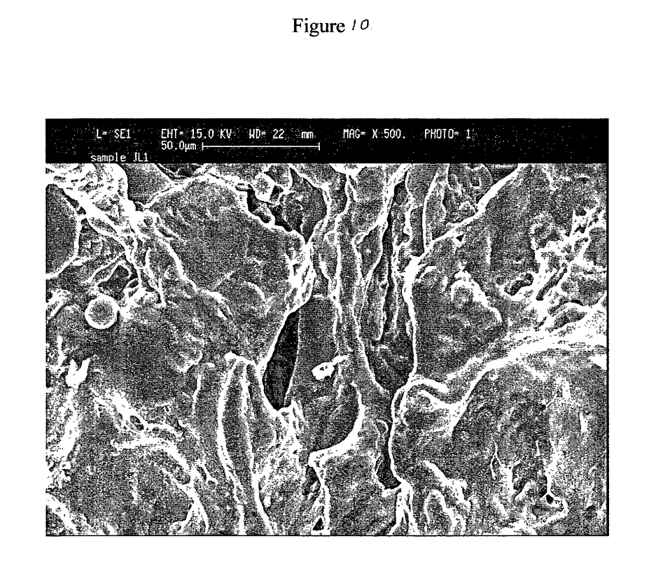

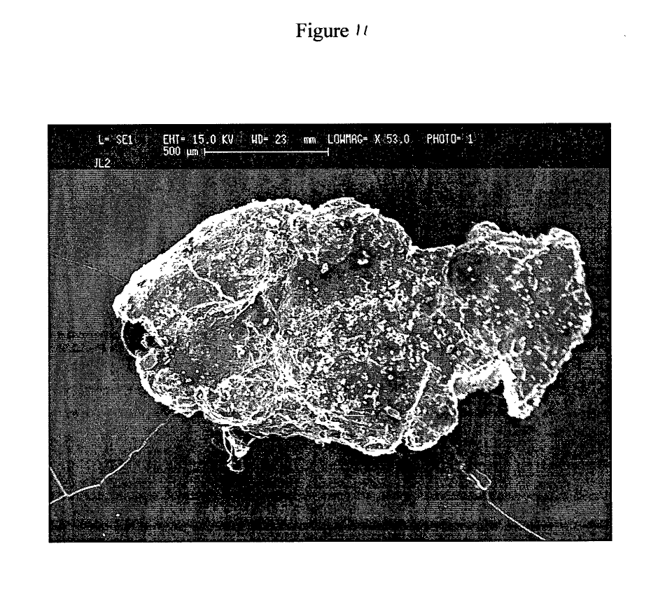

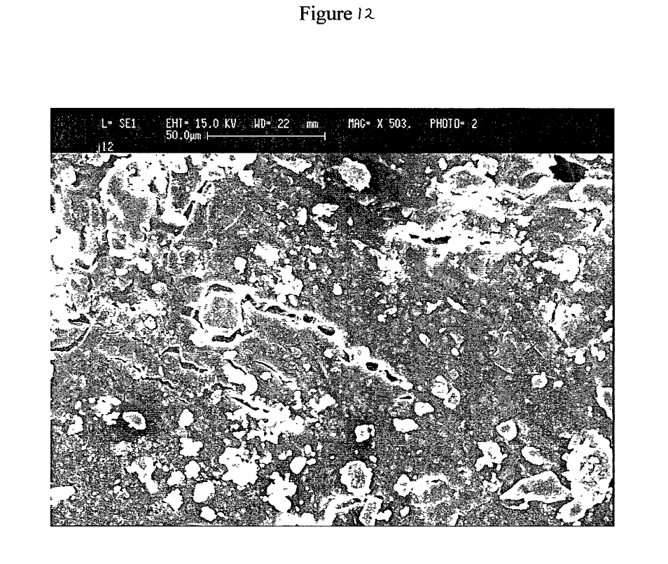

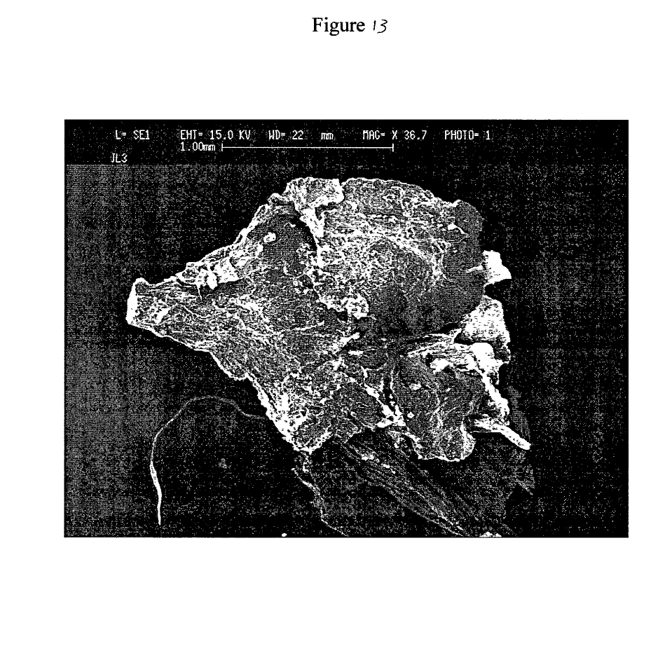

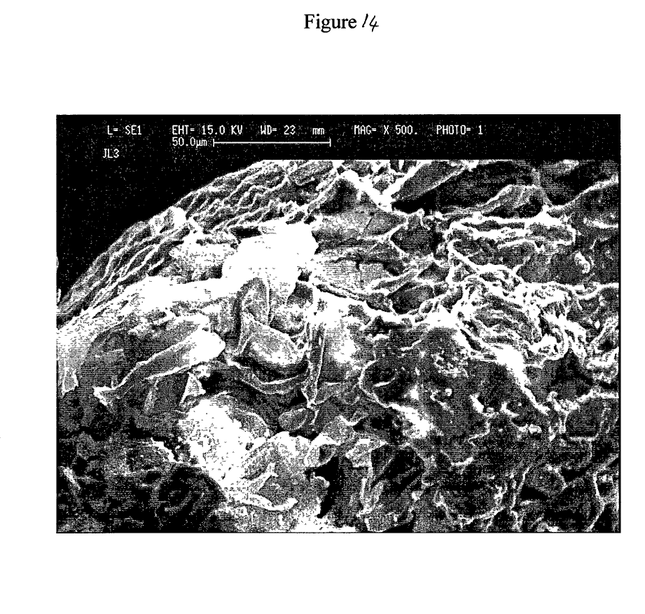

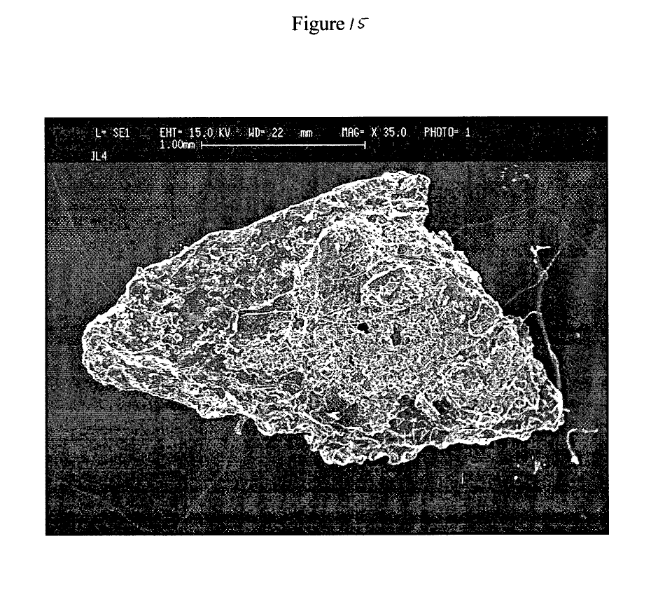

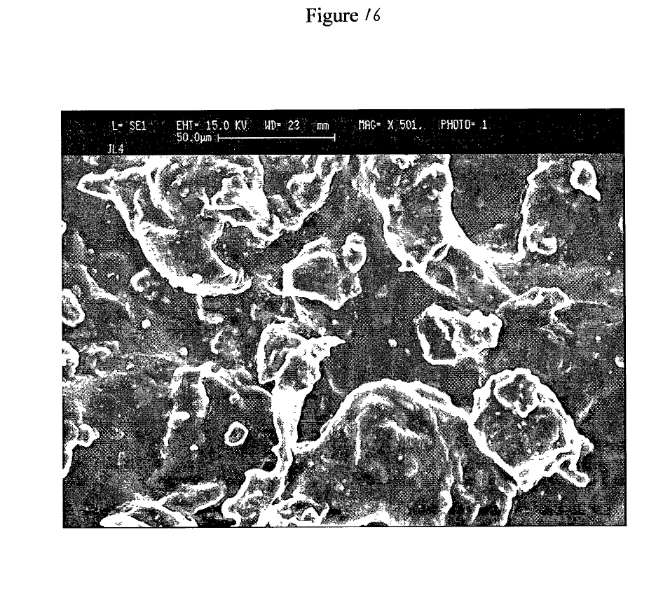

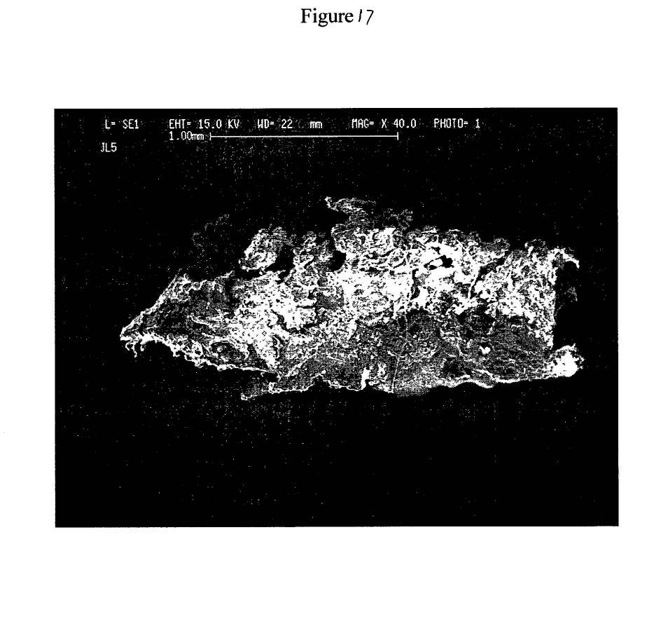

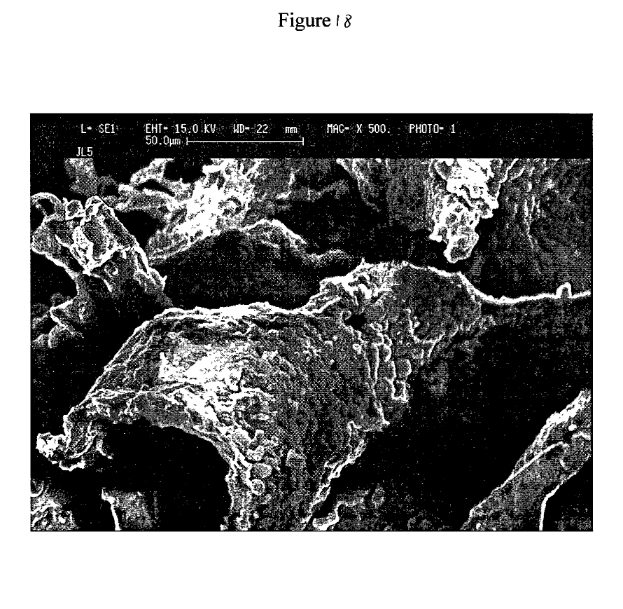

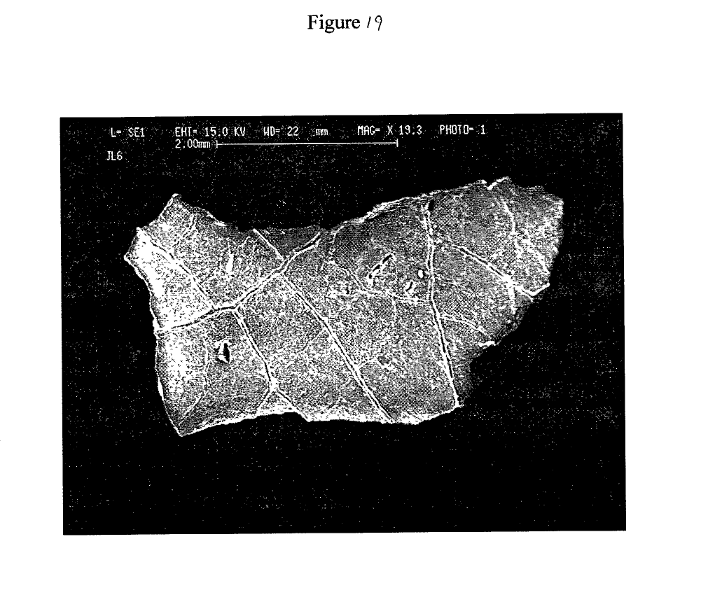

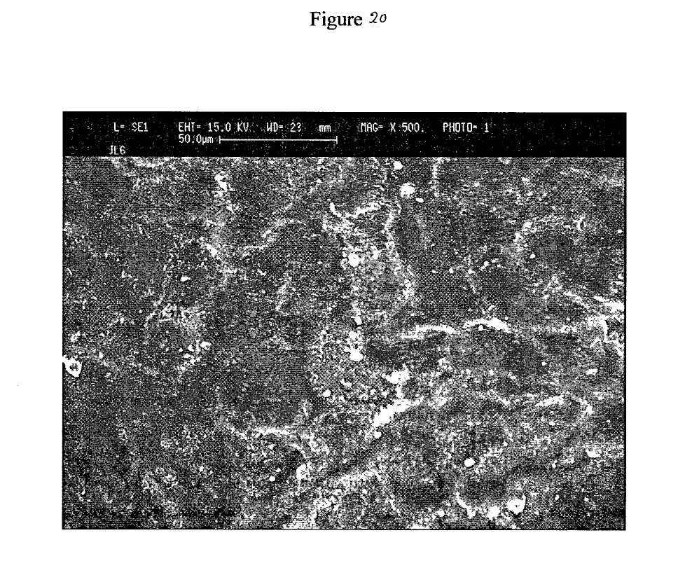

I next examined six of the particles using X-ray (Energy Dispersive X-ray, EDX) spectroscopy (Link ISIS™ Analyzer, Oxford Instruments, Concord, Mass. 01742), attached to a scanning electron microscope. This technique can quantitatively identify the majority (but not all) elements present in concentrations greater than approximately 1% on the surface area under examination. The software program and graphical output record the relative amounts of each of the selected elements. The equipment was available at the Center for Electron Microscopy and Microanalysis, University of Southern California, and was operated by Mrs. A. Thompson. For each particle, we chose at least 3 areas for sampling. I also obtained both a low power and a high power scanning electron micrograph of each of the six particles. The data can be summarized as follows: The particles differed from each other in their overall topography. Some had ruffled surfaces, while others were more smooth. They all appeared as conglomerates but differed in having irregularly shaped, curved, plate-like or ribbon-like protrusions. Several of the particles also contained numerous bright spots indicative of areas that were emitting relatively higher levels of x-rays than the rest of the particle. (This occurred in spite of all the particles being carbon coated prior to the examination). Such details can be seen in the series of photographs provided in FIGS. 9-20. The x-ray spectroscopic patterns also differed markedly for each of the particles, and to a lesser, but still very significant extent, within each of the particles. The following table records the results from each of two areas in the six particles.

TABLE 1
X-Ray Spectroscopy of Patient Derived Particles

| | | Relative Percentage of Elements in the Particles | | | | | | | | |
|---|---|---|---|---|---|---|---|---|---|---|
| Particle Number | Region | Na | Mg | Al | Si | P | S | Cl | K | Ca |
| 1 | a | 4.5 | * | 22.6 | 0.7 | 2.0 | 3.9 | 46.2 | 20.2 | * |
| | b | 7.4 | * | 14.4 | 0.1 | 0.6 | 3.5 | 59.9 | 14.0 | * |
| 2 | a | 4.5 | 14.3 | 9.0 | −1.9 | 1.6 | 17.9 | 0.0 | 5.1 | 49.5 |
| | b | 2.6 | 11.8 | 23.1 | 4.8 | −1.2 | 8.8 | 0.3 | 6.8 | 42.0 |
| 3 | a | −0.4 | 7.1 | 10.6 | 0.7 | 3.8 | 3.1 | −1.2 | 8.4 | 68.0 |
| | b | 2.0 | 2.4 | 1.5 | 4.1 | 1.1 | 4.4 | −0.4 | 10.9 | 74.0 |
| 4 | a | 2.4 | 6.1 | 15.6 | 6.6 | 2.0 | 7.9 | 15.8 | 8.7 | 34.9 |
| | b | −0.4 | 4.6 | 29.0 | 7.2 | −0.5 | 5.0 | 2.3 | 21.1 | 31.5 |
| 5 | a | 1.6 | 6.7 | 37.6 | 10.1 | 1.6 | 4.2 | 10.1 | 9.0 | 19.2 |
| | b | 2.4 | 4.4 | 21.0 | 5.2 | 1.2 | 4.4 | 14.0 | 9.0 | 38.5 |
| 6 | a | −1.8 | 5.7 | 3.1 | 0.4 | −1.6 | 8.3 | 4.8 | −3.1 | 84.2 |
| | b | 1.4 | 3.6 | 18.0 | 3.0 | 1.5 | 2.2 | 10.6 | 3.3 | 56.6 |

Na, sodium; Mg, magnesium; Al, aluminum; Si, silicon; P, phosphorus; S, sulfur; Cl, chloride; K, potassium; Ca, calcium.
* Not recorded with this particle since the amount was negligible.

Examples of inter-particle differences include the lack of detectable calcium in particle 1, yet this was the predominant mineral in particles 3 and 6. Chloride was the predominant component in particle 1, but was essentially absent in particles 2 and 3. Examples of intra-particle differences can be seen in the relative percentages of aluminum in particles 2, 3 and 6; magnesium, silica and phosphorous in particle 3; calcium in particles 5 and 6; etc.

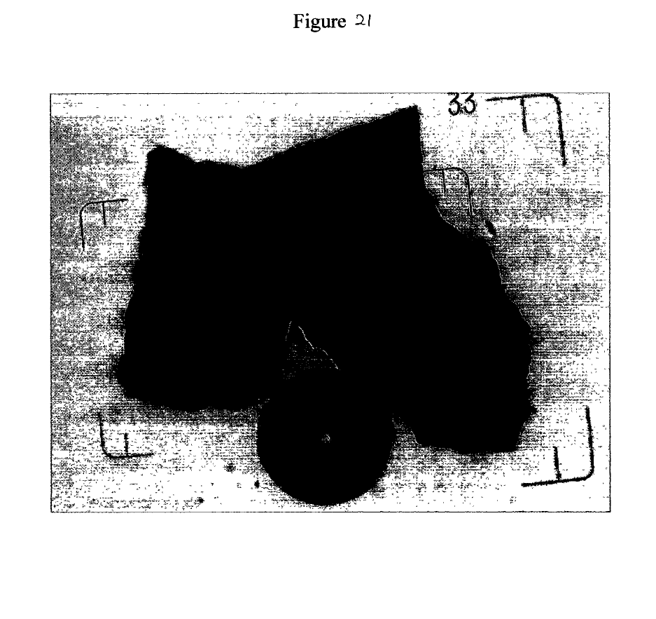

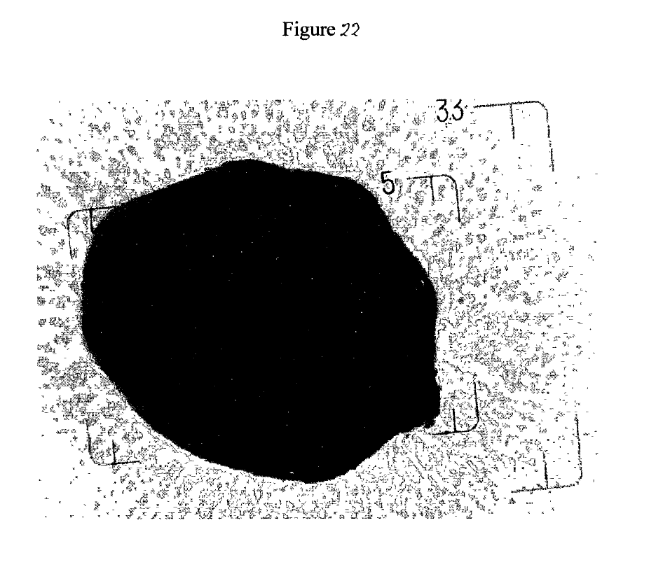

I had earlier tested some different particles for metabolic activity. When placed in tissue culture medium, some of the particles displayed clearly perceptible rapid vibrations. Moreover, gas bubbles were seen forming beneath some of the particles. For example, the same particle photographed in FIGS. 2-4, had joined with another magnetic particle. Over a 30 minute period, I could clearly see a gas bubble forming (FIG. 21). To another particle, I added a solution of the tetrazolium salt MTT ((Thiazole, Sigma catalog M5665) diluted in medium. Over several hours, the particle was able to reduce (add electrons to) the MTT to form blue formazan (FIG. 22). With another particle, I could demonstrate the reduction of ascorbic acid to calcium oxalate-like crystals.

In an effort to confirm that these were calcium oxalate crystal, I added some glacial acetic acid to check on their insolubility (Proia and Brinn, 1985). Immediately, I observed a frenzy of activity both in the fluid and the particle. Specifically, the particle disrupted into numerous highly motile, very small (approximately 0.2-0.5 um diameter), near-spherical components. I could watch these components move erratically to and from the originating particle. After seemingly a series of repelling contacts with the remaining particle and with other released components, small groups of these components would adhere together to form small clusters. Occasionally a newly formed cluster would undergo a violent disruption, only to reform again at another location. The movements only ceased when the acetic acid had evaporated. I could achieve the same effects with other particles using acetone, acetic ester, acetonitrile, methanol, propanol and alcohol. It did not occur with 1N sulfuric acid, perchloric acid or water. It was also much more marked when the fluid was exposed to air on a glass slide than when the fluid was used in a closed tube.

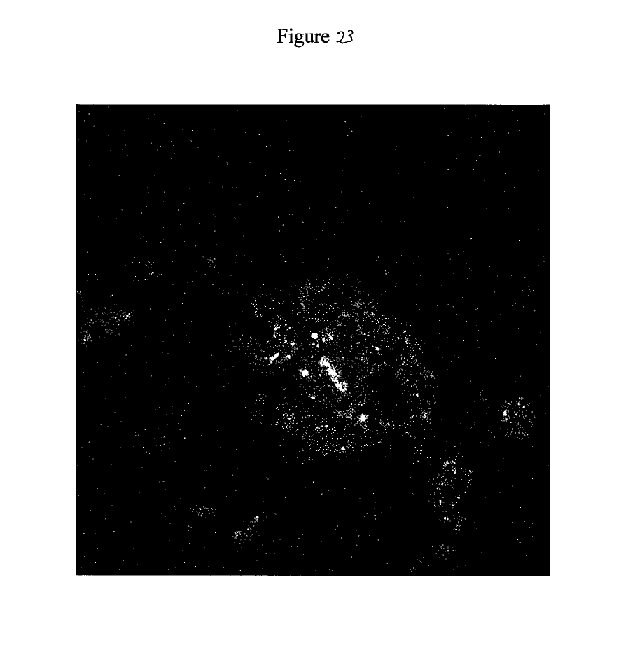

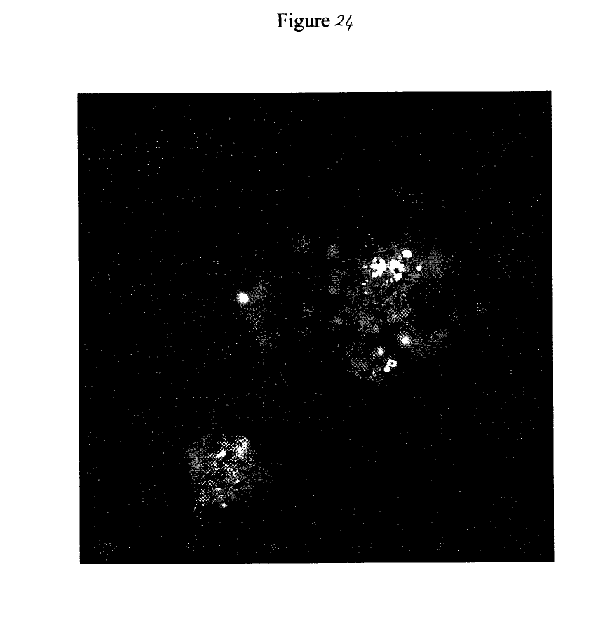

I had been working with humic acid preparations and had noted morphological and functional similarities between the fragments present in several humic acid preparations and the pigmented ACE-pigments developing in stealth virus cultures. I extended this study to include auto-fluorescence analysis of humic acid. The material used was mainly a particulate preparation of humic acid mixed with volcanic ash (Boost 77, dietary supplement, Morningstar Minerals, Farmington, N. Mex. 87499). Using both a regular fluorescent microscope and the confocal microscope at the City of Hope Medical Center, I could readily see strong and sustainable auto-fluorescence. Although the auto-fluorescence of the humic acid particles was more restricted than with the patient's particles, I could easily discern that they too were comprised of a conglomerate (or to borrow a term from geology, a breccia) of smaller irregularly shaped components. Examples of the bright, punctuate auto-fluorescence seen with humic acid particles are shown in FIGS. 23 and 24.

I also used x-ray spectroscopy to test the composition of the humic acid preparation. Two readings gave comparable, although slightly differing results, with a predominance of aluminum and the presence of iron (Table 2). The later was expected since I was able to show magnetic attraction some of the humic acid material. The majority of the humic acid particles could also be moved with an electrostatic force.

TABLE 2
X-ray Spectroscopy of a Humic Acid Preparation
Relative Percentage of Elements in the Humic Acid

| Na | Mg | Al | Si | P | S | C1 | K | Ca | Ti | Fe |
|---|---|---|---|---|---|---|---|---|---|---|
| 0.7 | 5.2 | 50.7 | 11.4 | 0.3 | 0.6 | −0.1 | 2.0 | 17.4 | 2.0 | 9.8 |
| 1.4 | 7.6 | 44.1 | 11.2 | 0.3 | 0.4 | 0.1 | 2.8 | 18.3 | 2.1 | 11.6 |

The abbreviations for the metals are those used in Table 1 with the addition of Ti for titanium and Fe for iron.

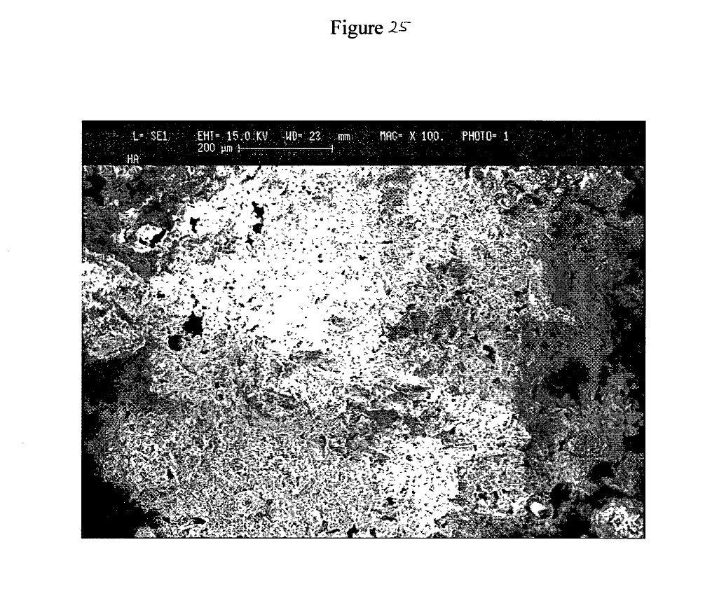

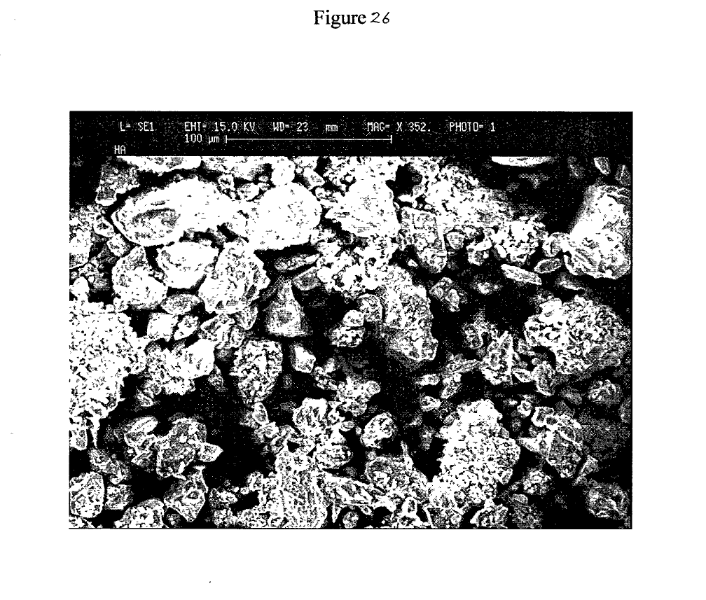

By scanning electron microscopy, the humic acid particle was clearly also composed of a conglomerate (or to borrow a word from geology, a breccia) of many finer components (FIGS. 25 and 26).

Based on the similarities of humic acid with the patient derived particulate material, I proceeded to expose humic acid particles to acetic acid and related oxidizing chemicals. I observed the same dramatic phenomenon as seen with the patient derived particles. Moreover, I could confirm the observations with each of three independent preparations of humic acid (moor). The liquid became agitated as it interacted with the humic acid particles. More striking, the humic acid particles dissociated into rapidly and erratically migrating very small components. As with the patient-derived particles, these released components showed both attractive and repulsive forces to the larger particles and to other released components. Small groups of components would gradually form into more stable clumps. For controls, I have used several powered herbal mixtures and inert iron filings, none of which was activated by the aldehydes or alcohols.

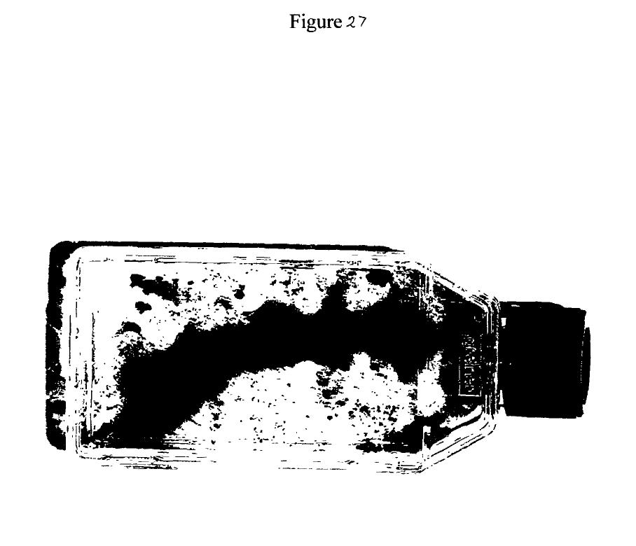

A series of additional studies was performed with a soluble, particulate free, preparation of humic acid also provided by Morningstar Minerals. I was able to precipitate very fine flocculent material from a 1:50 dilution in water of the soluble humic acid preparation by adding a 1:5 concentration of acetic acid in a T25 tissue culture flask. The fine particles formed soon begin to engage in an active streaming process of aggregation with eventual condensation of virtually all of the material into a mat covering less that half the bottom surface of the flask. An example is shown in FIG. 27. The aggregates could be easily re-dispersed into fine unattached bodies by vigorous shaking of the flask. This would invariably lead to the release of gas bubbles. I could show further that this aggregation process was promoted by exposure to light and/or heat.

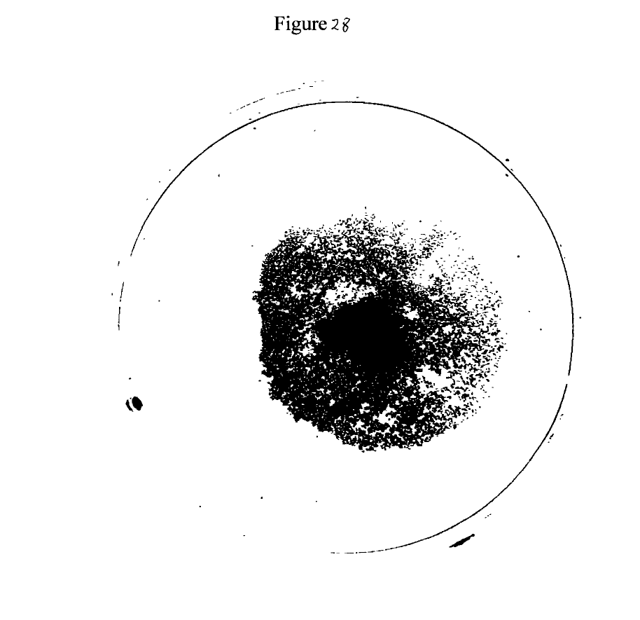

What became striking in these studies was the apparent active process involved in the coalescence of the humic acid material. it clearly was involving an inter-particle attraction force. This attraction force could also be demonstrated with insoluble humic acid particles gently allowed to float freely on the surface of water in a Petri dish. As various floating particles came near each other, I could easily distinguish between three possible outcomes. First, the particles would be indifferent to each other's presence and simply float by undisturbed. Second, there would be a distancing and apparent repulsion from each other. The third, and least likely but most striking occurrence, was for the two particles to assume new alignments closely approached and then to quickly snap together. The combined particle would proceed on its way, ignoring and repelling some of floating particles, yet binding actively to others. Eventually, the floating particles would join together in a selective, complex arrangement, that would typically form a small inner circle in the middle of the Petri dish, as shown in FIG. 28.

I could exclude the involvement of magnetic or electrostatic forces in this aggregation process. Thus neither a magnet or an electrostatically charged plastic ruler (or Styrofoam cup) had any appreciable effect on the inter-particle attachment process. Moreover, it was clear that the observed aggregation was not simply based on a two way (yes/no or positive/negative) phenomenon. Rather, the observed attachments were more discriminatory and selective, with a frequency of active inter-particle binding well less than 50%. This type of multiple selectivity also excluded gravity that would have acted in a non-discriminatory manner.

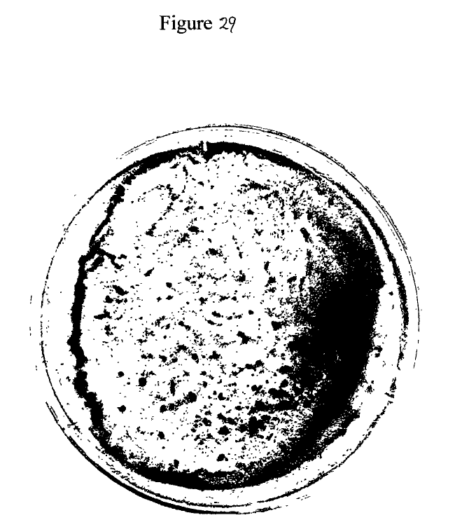

In another demonstration of the phenomenon I observed that the humic acid material that sank to the bottom of water or a balanced salt buffered solution, would similarly migrate from being uniformly dispersed across the bottom of the Petri dish or flask, to form into distinct linear and solid clusters. This partitioning and clumping process could be enhanced by exposing the Petri dishes and flasks to sunlight. An example of an irregular pattern resulting from an evenly dispersed suspension of humic acid in a Petri dish left out in the sun is provided in FIG. 29. Using a magnifying glass or viewing with a microscope, I could observe periodic movements of both small and large particles. Some of the particles viewed under the microscope were clearly attracted to light and could be led from one region of the flask to another by progressively moving the area illuminated by the microscope light.

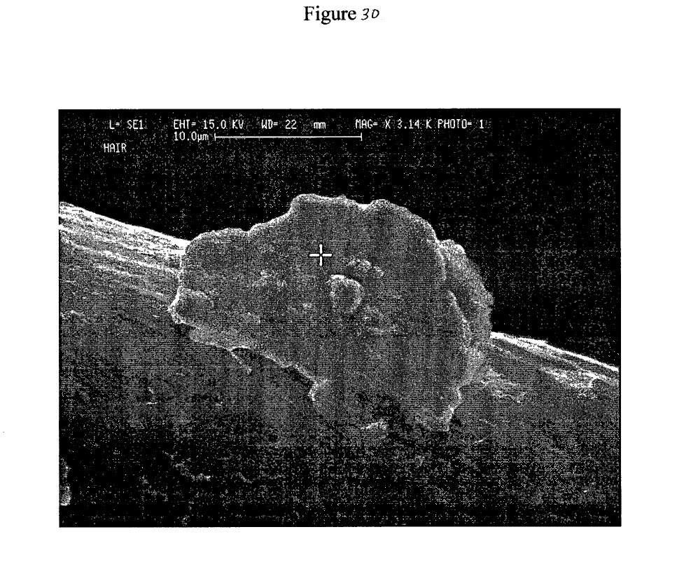

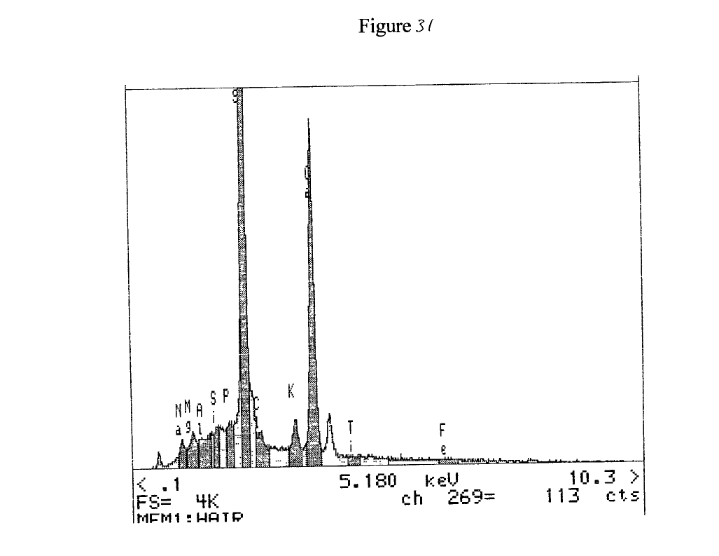

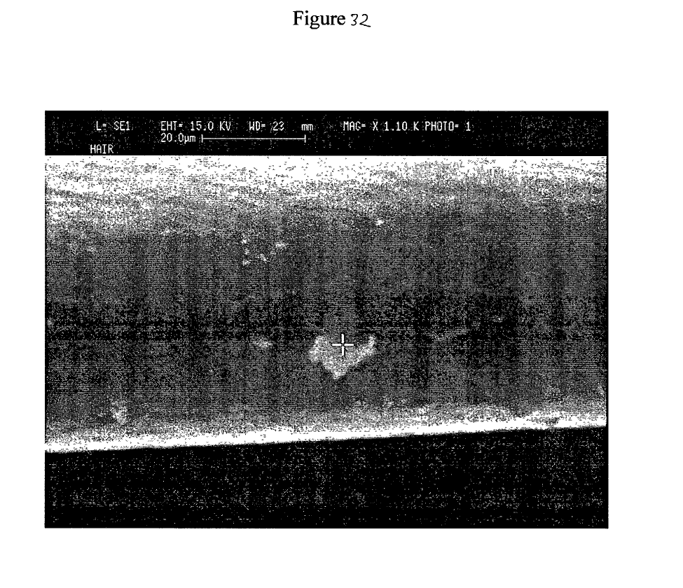

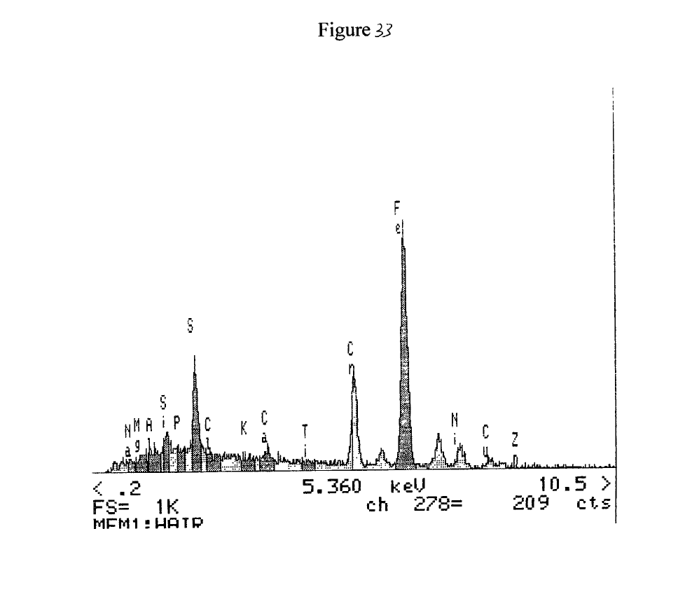

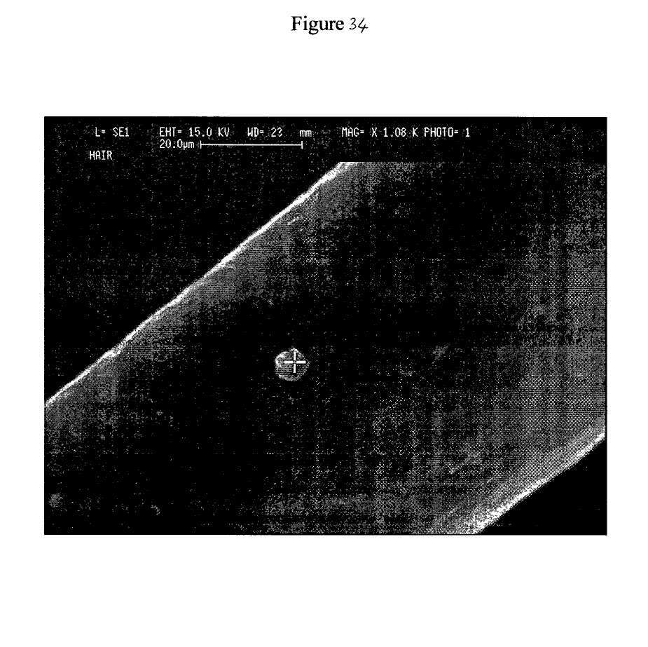

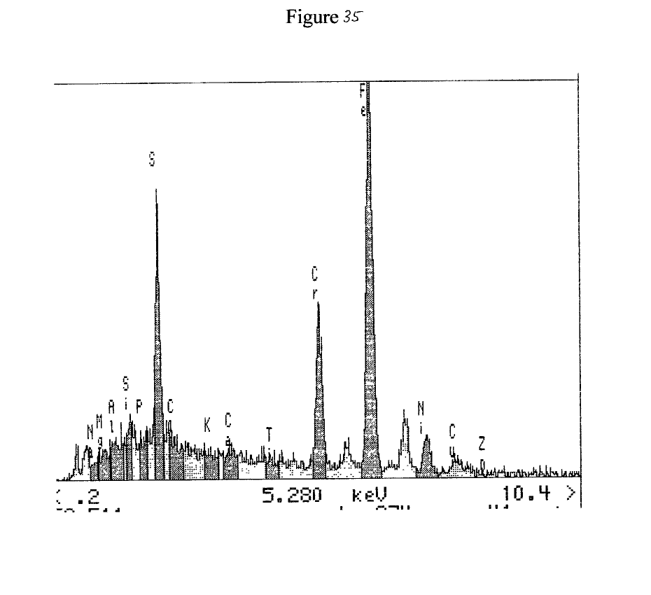

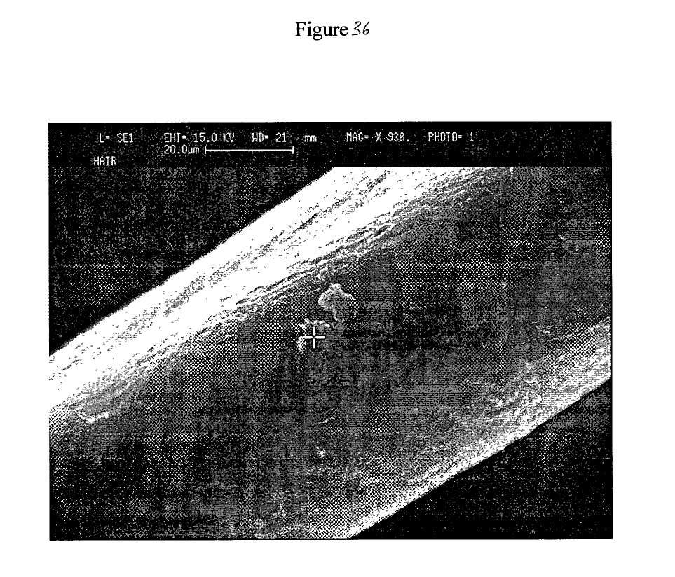

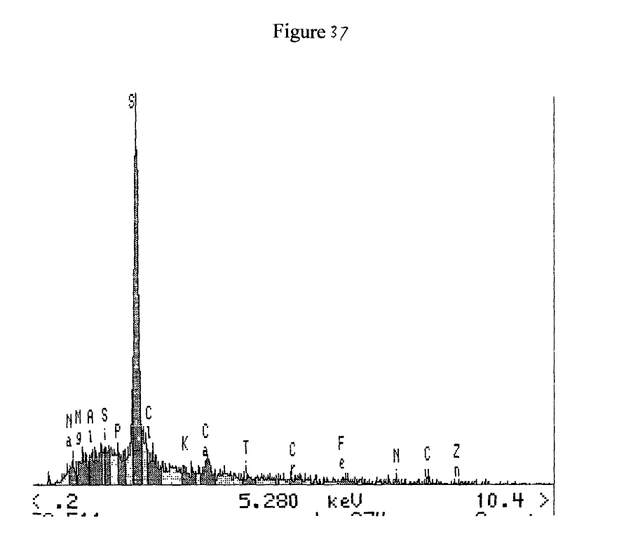

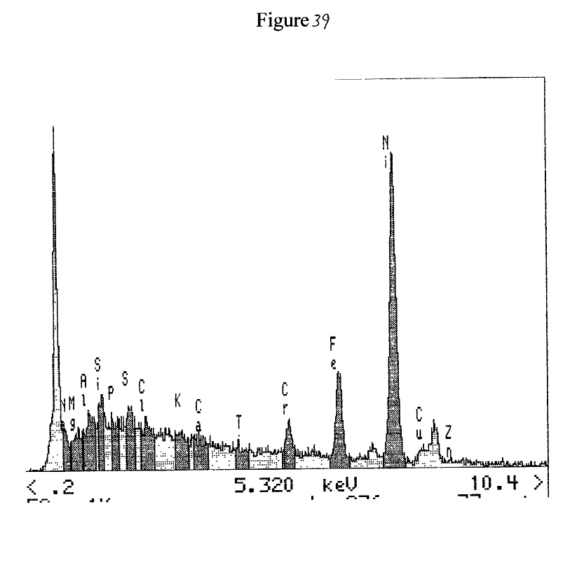

Lastly, I decided to use x-ray spectroscopy to determine the composition of the very small particles that I had seen attached to the hair strands of various stealth virus infected patients. Each of 5 separate particles examined on a small sampling of pubic hair strands provided by a patient, showed a unique composition when examined for various elements. A scanning electron micrograph of each of the 5 particles and the accompanying x-ray spectroscopy results for each particle are provided in FIGS. 30-39. The abbreviations for the metals are those used in Tables with the addition of Cr for chromium; Ni for nickel; Cu for copper; and Zn for Zinc. The unlabeled peaks in Figure are secondary emissions (L shell) from one or other of the labeled elements.

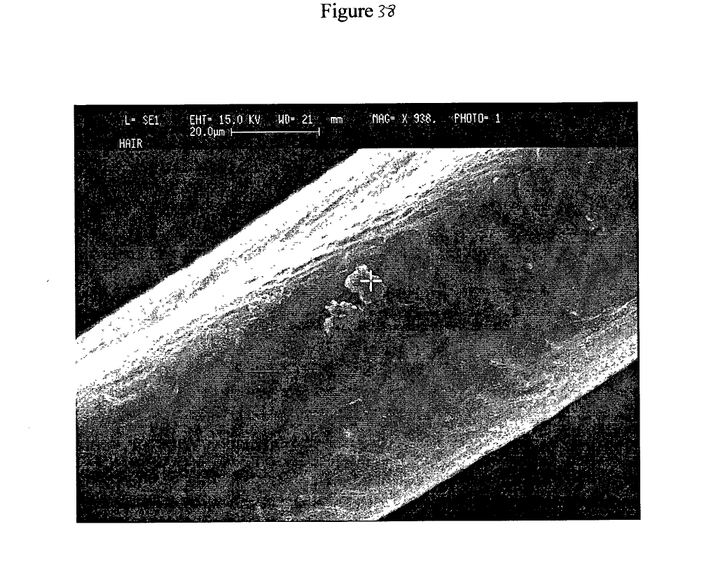

One of the hair attached particles showed essentially only sulfur; another predominantly sulfur and calcium. Two particles had relatively less sulfur but with easily detectable chromium and iron, in differing ratios, along with a little nickel and copper; the fifth showed predominantly nickel, with easily measurable iron and chromium. Even closely adjacent particles such as the two particles seen in FIGS. 3 and 38 showed remarkably different compositions.

From these results, I concluded that the striking differences in the compositions showed that each particle must have a propensity to incorporate molecules with a matching content. Certainly the data argue against random aggregation process.

This means that I have discovered a heretofore unidentified physical force that enables molecules to conglomerate into macroscopic particles. This binding force is presumably based on a form of inter-molecular recognition. In conventional scientific thinking, the only forces of attraction operating on particles are electrical, including, electrostatic; magnetic; and gravitational. Particles can also be forced into physical association if they are concentrated by being partitioned into either the aqueous or non-aqueous phase of a liquid mixture. The novel force identified in this research differs fundamentally from all of these forces. Specifically, it is multi-dimensional. Based on the composition of particles attached to hair, it is also seemingly selective for the pairing of molecules with generally similar elements. I am, therefore, tentatively using the term “Selective Mineral Affinity Force” or SMAF.

SMAF is defined as the force of attraction between similar elements contained within organic molecules that can lead to aggregation of the molecules into particulate structures, and that is distinguishable from magnetic and electrostatic forces. SMAF is contained in ACE-pigments and humic acid particles. It is likely to be operating in many other situations. This novel force will likely provide a more selective alternative to the many and varied uses of magnetic and electrical forces in industrial applications. These potential uses can be achieved even in the absence of a detailed understanding of the actual atomic basis of the SMAF operating between interacting molecules. In this regard, the exact nature of both magnetic and gravitational forces are not yet fully understood by physicists.

I have previously disclosed that ACE-pigments can be activated by various forms of electromagnetic radiation, including gamma rays, x-rays and visible light. They are then better able to provide chemical energy, such as that required to convert MTT to formazan, and ascorbic acid to oxalate. In the present application I showed that humic acid particles can also respond to light. Both ACE-pigments and humic acid are potentially useful in converting electromagnetic energies into chemical energy. On a cellular level, ACE-pigments, and quite possibly administered humic acid, may be helpful in resuscitating cells with impaired mitochondria, such as for example, stealth virus infected cells. On a larger scale, these materials could be used to trap some of the sun's energy and converting it to chemical energies required for major bio-synthetic reactions. Thus, even without fully understanding the nature of the electromagnetic to chemical energy conversion, it will be apparent to those knowledgeable in the area that both ACE-pigments and humic acid will be useful as chemical energy producing materials. Conversely, they can potentially provide sensors for the detection of electromagnetic energies.

Exposure of ACE-pigments and humic acid particles to certain aldehydes and alcohols provides considerable energy in the form of the rapid movements of the numerous components released by these chemicals. This energy could also be harvested for a variety of uses.

The concept that energy emitting, disease related materials is present in and can be released from stealth virus infected patients has yet another association. Certain individuals, commonly known as healers, can use their hands to sense a patients state of health. As they pass their hands over a sick person, they can experience a force emanating from the patient. Some healers can demonstrate slight deviations in the vertical alignment of a pendulum made from silica or other minerals, when hung close by a patient. It occurred to me that these phenomena may be related to SMAF. I, therefore, sought the input from a person experienced in the art of using her hands to access the well being of others. I asked if she could sense the presence of humic acid of the remaining particles provided by the patient. On each of two occasions, she definitely felt an attraction as her hands came near the humic acid and, to an even greater extent, near the patient's particles. As she pulled her hands up, she could feel a release of the attraction. She did not feel any of the same sensation when asked to move her hands nearby an electrostatically charged plastic ruler, a magnet, or capsules of herbal product. I can envision providing humic acid and patient derived particulate materials to help healers improve their art, and to help engineers devise more sensitive methods of SMAF detection.

The principles, preferred embodiments and modes of operation of the present invention have been described in the foregoing specification. The invention which is intended to be protected herein, however, is not to be construed as limited to the particular forms disclosed, since they are to be regarded as illustrative rather than restrictive. In particular, the claim for SMAF is not restricted to only ACE-pigments and humic acid but is intended to cover other substances in which this force is acting. Similarly, the term humic acid is intended to cover structurally related organic material that shares the property with humic acid of being held together as aggregates by SMAF. Additional advantages and modifications will readily occur to those skilled in the art. Variations and changes may be made without departing from the spirit of the invention.

What I claim:

1. The uses of ACE-pigments, defined as the auto-fluorescent mineral containing particulate material produced by cells in response to a mitochondria damaging viral infection, as a source of a novel form of energy termed SMAF, being defined as the non-magnetic and non-conventional electrostatic force of attraction between similar elements contained within organic molecules that can lead to aggregation of the molecules into particulate materials.

2. The uses of humic acid, defined as the auto-fluorescent, mineral containing particulate material produced by decaying vegetation, as a source of a novel form of energy termed SMAF, being defined as the non-magnetic and non-conventional electrostatic force of attraction between similar elements contained within organic molecules that can lead to aggregation of the molecules into particulate materials.

3. The uses of the novel form of energy termed SMAF, defined as the force of attraction between similar elements contained within organic molecules that can lead to aggregation of the molecules into particulate materials, as an alternative to magnetic, electrical and gravity forces in applications where these alternative attraction forces are currently being used.

4. The uses of ACE-pigments, defined as the auto-fluorescent, mineral containing particulate material produced by cells in response to a mitochondria damaging viral infection, as a means of converting electromagnetic energy into chemical energy.

5. The uses of humic acid, defined as the auto-fluorescent, mineral containing particulate material produced by decaying vegetation, as a means of converting electromagnetic energy into chemical energy.

6. The use of auto-fluorescence in the detection and energy excitation and emission characteristics of differing sources of ACE-pigments and humic acid.

7. The use of x-ray spectroscopy in the detection and energy excitation and emission characteristics of differing sources of ACE-pigments and humic acid.

8. The use of chemicals chosen from the group of acetic acid, acetone, acetonitrile, ethyl acetate, methanol, propanol and ethyl alcohol to cause the disruption of ACE-pigments and humic acid into highly energized, motile components.

9. The use of re-aggregating particles of ACE-pigments and humic acid to demonstrate and to characterize the novel form of energy as defined in claim 1.
